(19) United States
(12) Patent Application Publication
(10) Pub. No.: US 2026/0065538 A1
(43) Pub. Date: Mar. 5, 2026

(54) COMPUTER-IMPLEMENTED METHOD OF GENERATING A GLYPH REPRESENTATION FOR USE IN A RENDERING PROCESS

(71) Applicant: Siemens Healthineers AG, Forchheim (DE)

(72) Inventors: Felix DINGELDEY, Erlangen (DE); Lars BOHN, Neunkirchen am Brand (DE)

(73) Assignee: Siemens Healthineers AG, Forchheim (DE)

(21) Appl. No.: 19/314,326

(22) Filed: Aug. 29, 2025

(30) Foreign Application Priority Data
Aug. 30, 2024 (DE) 10 2024 208 244.9

(51) Int. Cl. G06T 11/20; G16H 30/40

(52) U.S. Cl. CPC G06T 11/23; G16H 30/40

(57) ABSTRACT

A computer-implemented method of generating a further glyph representation for use in a rendering process, comprises: determining a first plurality of locations in the further glyph representation corresponding with a respective plurality of source locations in a source glyph representation; setting respective first indices for the first plurality of locations based on values associated with the respective plurality of source locations; setting, for each location of a further plurality of locations in the further glyph representation, respective second indices based on a relationship between the location and one or more source locations, wherein the further plurality of locations include the first plurality of locations and a second plurality of locations; and storing the respective first and second indices in association with respective locations. A computer-implemented method of rendering a glyph using the further glyph representation is also described.

### CROSS-REFERENCE TO RELATED APPLICATION(S)

The present application claims priority under 35 U.S.C. § 119 to German Patent Application No. 10 2024 208 244.9, filed Aug. 30, 2024, the entire contents of which are incorporated herein by reference.

### FIELD

One or more example embodiments of the present invention relate to a computer-implemented method for generating a glyph representation for use in a rendering process and a computer-implemented method of using such a glyph representation to render a glyph. One or more example embodiments of the present invention also relate to a set of machine-readable instructions for implementing the method, a non-transitory machine-readable medium comprising such instructions and an apparatus for implementing the method.

### BACKGROUND

In computer-implemented rendering, such as in text rendering, glyph atlases are commonly used to achieve better performance. A glyph atlas may be a 2D memory buffer storing pre-rendered glyphs from which output text can be rendered glyph-by-glyph.

In some examples, it may be desirable to render an output, such as output text, with a visual effect applied. For example, the legibility of text may suffer when it is rendered on an inhomogeneous background, for instance, if the color of the text and the background have a similar hue or brightness. This may occur commonly in medical imaging applications where text is overlayed on top of a medical image. In such applications, to increase text legibility, an effect may be applied to the text. For example, the text may be rendered with a shadowing effect, such that the inhomogeneous background is covered by a shadow with the text rendered in its regular foreground color and style on top of the shadow. However, while such effects can improve the legibility of text in certain applications, rendering with such effects may reduce the advantages of using a glyph atlas, since the computational and/or memory costs of using pre-rendered glyphs may be reduced.

### SUMMARY

According to a first aspect of one or more example embodiments of the present invention, there is provided a computer-implemented method of generating, based on a source glyph representation, a further glyph representation for use in a rendering process, the method comprising: determining a first plurality of locations in the further glyph representation corresponding with a respective plurality of source locations in the source glyph representation and setting respective first indices for the first plurality of locations based on values associated with the respective source locations; setting, for each given location of a further plurality of locations in the further glyph representation, wherein the further plurality of locations comprises the first plurality of locations and a second plurality of locations, respective second indices based on a relationship between the given location and a source location in the source glyph representation; and storing the first indices and the second indices in association with the respective locations.

The setting the respective first indices of the first plurality of locations based on values associated with the respective source locations may comprise applying a quantization process to the values associated with the respective source locations.

The relationship may comprise a geometric relationship between the given location and one or more of the one or more source locations. The geometric relationship may comprise a distance metric.

The setting the second indices may comprise performing a visual-effect calculation based on the relationship between the given location and the one or more source locations in the source glyph representation.

The visual-effect calculation may comprise a shadow-effect calculation and/or a blurring-effect calculation.

The first indices and the second indices may be stored in a number of bits per location in the further glyph representation which is less than or equal to a number of bits per location in which the values associated with the respective source locations are stored in the source glyph representation.

The values associated with the respective source locations may be visual parameter values, such as grayscale or color values.

According to a second aspect of one or more example embodiments of the present invention, there is provided a computer-implemented method of rendering a glyph, comprising, using a glyph representation generated by a method according to the first aspect of one or more example embodiments of the present invention: performing a background rendering process, comprising using the second indices of the glyph representation to determine background visual parameter values for a plurality of locations in a background region corresponding to the further plurality of locations in the glyph representation; and performing a foreground rendering process, comprising using the first indices of the glyph representation to determine foreground visual parameter values for a plurality of locations in a foreground region corresponding to the first plurality of locations in the glyph representation.

The determining the background visual parameter values for the plurality of locations in the background region may be based on a pre-determined mapping between the second indices and background visual parameter values; and/or the determining the foreground visual parameter values for the plurality of locations in the foreground region may be based on a pre-determined mapping between the first indices and foreground visual parameter values.

The method of rendering a glyph may be applied in a medical rendering process, and the method may comprise: selecting the pre-determined mapping between the second indices and the background visual parameter values and/or the pre-determined mapping between the first indices and the foreground visual parameter values based on a property of the medical rendering process.

According to a third aspect of one or more example embodiments of the present invention, there is provided a machine-readable medium comprising one or more glyph representations generated by a method according to the first aspect of one or more example embodiments of the present invention.

According to a fourth aspect of one or more example embodiments of the present invention, there is provided a set of machine-readable instructions which when executed by a processor cause a method according to the first aspect or the second aspect of one or more example embodiments of the present invention to be performed.

According to a fifth aspect of one or more example embodiments of the present invention, there is provided a non-transitory machine-readable medium comprising a set of machine-readable instructions according to the fourth aspect of one or more example embodiments of the present invention.

According to a sixth aspect of one or more example embodiments of the present invention, there is provided apparatus comprising: a processor; and a storage comprising one or more glyph representations generated by a method according to the first aspect of one or more example embodiments of the present invention and/or a set of machine-readable instructions according to the fourth aspect of one or more example embodiments of the present invention.

### BRIEF DESCRIPTION OF THE DRAWINGS

The present invention will now be described, by way of example only, with reference to the following figures, in which:

FIG. 1 is a flow chart illustrating a computer-implemented method of generating, based on a source glyph representation, a further glyph representation for use in a rendering process;

FIG. 2 is a flow chart illustrating a computer-implemented method of rendering a glyph using a glyph representation generated by a method illustrated by FIG. 1;

FIG. 3 is a schematic drawing illustrating aspects of an example method for generating a glyph representation; and

FIG. 4 is a schematic drawing showing an apparatus for performing example methods described herein.

### DETAILED DESCRIPTION

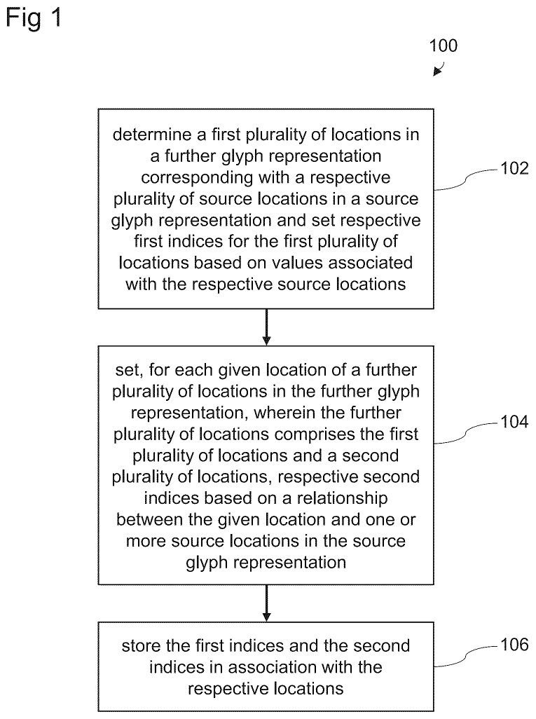

FIG. 1 is a flowchart illustrating an example computer-implemented method 100 of generating, based on a source glyph representation, a further glyph representation for use in a rendering process.

The source glyph representation may be a representation of a particular glyph in a glyph atlas, which may be referred to as a source glyph atlas. The source glyph atlas may comprise one or more other source glyph representations. For example, the source glyph atlas may comprise a plurality of respective representations of different Latin or Arabic characters in a particular style and size. Each particular character in a particular style and size, for example, the Latin letter “A” in Arial font, 11 points, may be referred to as a glyph. The representation of a particular glyph in the source glyph atlas may be referred to as a source glyph representation. Each source glyph representation in the source glyph atlas may represent a respective glyph with an anti-aliasing effect applied thereto.

The source glyph representation comprises a plurality of source locations, for example, a set of pixels, with each location having one or more values stored in association with it. These values may be referred to herein as source values. The source values may be visual parameter values, such as grayscale or color values. For example, in some examples, the source glyph representation may be a grayscale representation of the glyph and may comprise a plurality of pixels each with an associated grayscale value. In other examples, the source glyph representation may be color representation of the glyph and may comprise a plurality of pixels each with one or more associated color values, for example, a respective R, G and B value per pixel.

The plurality of source locations in the source glyph may be a grid of pixels of any other suitable size depending on the resolution of the source glyph representation and the size of the glyph being represented. The grid of pixels may be regularly or irregularly shaped. In some examples, the plurality of pixels representing the source glyph representation may be referred to as the “black box” of the source glyph representation.

The further glyph representation generated by the method 100 may be a representation of the glyph represented by the source glyph representation with a visual effect applied thereto. For example, the further glyph representation may represent the glyph with a background effect, such as a shadowing effect, e.g. a blurred shadow effect, applied thereto. The further glyph representation may form part of a further glyph atlas comprising one or more other further glyph representations. For example, each further glyph representation in the further glyph atlas may correspond with a source glyph representation in the source glyph atlas with the visual effect applied thereto.

The method comprises, at block 102, determining a first plurality of locations in the further glyph representation corresponding with a respective plurality of source locations in the source glyph representation.

The first plurality of locations may, for example, be a plurality of pixels in the further glyph representation which are determined to correspond with the pixels of the black box of the source glyph representation. These may be considered to be foreground pixels of the further glyph representation as they are pixels which represent the foreground text information corresponding to the original source glyph. The further glyph representation also comprises a second plurality of locations in addition to those determined to correspond with the source locations. The second plurality of locations may provide, for example, a margin of pixels around the pixels corresponding to the source pixels and may be used to accommodate a visual effect, such as a shadow effect. The number and position of the second plurality of pixels relative to the first plurality of pixels may depend on the desired visual effect to be represented, as is described by way of example below.

At block 102, the method also comprises setting respective first indices for the first plurality of locations based on values associated with the corresponding respective source locations.

As described above, the values associated with the respective source locations, that is, the source values, may be respective grayscale values for each of the pixels in the black box of the source glyph representation. These values may be stored in a plurality of bits in the source glyph representation, for example, 4 bits or 8 bits per pixel.

Setting the respective first indices for the first plurality of locations may comprise applying a quantization process to the source values. The quantization process may, for example, comprise defining quantization bins for the source values, and determining for each given location in the first plurality of locations, to which of the quantization bins the associated source value corresponds. Each quantization bin may be labelled with a value of the first index. In this way, the value of the first index for each given pixel can be determined by determining the quantization bin to which the associated source value corresponds. Examples of this are described in more detail below.

At block 104, the method comprises setting, for each given location of a further plurality of locations, respective second indices based on a relationship between the given location and a given source location in the source glyph representation. The further plurality of locations comprises the first plurality of locations, for example, the pixels corresponding to the black box of the source glyph representation, and the second plurality of locations, which, as described above, may be a second set of pixels outside of the black box of the source glyph representation providing a margin for representing a shadow or other visual effect.

The relationship used to set the second index for a given location of the further plurality of locations may, for example, be a geometric relationship between the given location and a location in the first plurality of locations corresponding with the given source location. The geometric relationship may, for example, be a distance metric, such as a Euclidean or a Manhattan distance. Setting the second index for the given location based on the relationship may comprise determining a value for the second index based on the relationship.

For example, the second index may be set based on a distance between a given pixel and one or more of the foreground pixels in the region of the further glyph representation corresponding to the black box of the source glyph representation. As an example, the second index for a given pixel may be set to represent a distance, e.g. a Manhattan distance in pixels, between the given pixel and a foreground pixel.

In some examples, the second index for a given pixel may be set based on respective relationships between the given pixel and more than one foreground pixel. For example, each pixel within a pre-determined region surrounding the given pixel may provide a contribution used to set the second index at the given pixel.

As will be described in more detail below, the second indices may be used to allow a visual effect, such as a background effect, to be rendered in the further glyph representation. For example, a background shadow effect may be provided which, for example, may decay in intensity with increasing distance from a foreground pixel. In other examples, a sharp outline or opaque background box may be provided by using the second indices.

In some examples, a visual-effect calculation, such as a shadow-effect calculation and/or a blurring effect calculation, may be performed to determine the second indices. As an example, setting the second index for a given pixel may comprise determining a visual parameter value, e.g. a shadow effect grayscale or similar value, for the pixel based on the relationship between the pixel and the one or more source pixels. In some such examples, the visual parameter value used to set the second index may be set based additionally on respective visual parameter values of the one or more source pixels. The second index may then be selected from the determined visual parameter value, for example, involving applying a quantization process in a similar manner to as described above in relation to the first index. An example of this will also be described in more detail below. In some such examples, where the second index is set based on a relationship between the given pixel and more than one foreground pixels, the value of the second index may be obtained by combining, for example, alpha blending, the contributions from each of the foreground pixels.

At block 106, the method comprises storing the first indices and the second indices in association with the respective locations.

The further glyph representation may comprise a plurality of bits of storage per location, for example, 8 bits per pixel. For each location, a first number of the bits may be used to store the first index and a second number of the bits may be used to store the second index.

The number of bits used to store each of the first index and the second index may depend on the method by which the first and second indices are set. For example, it has been observed by the inventors that many anti-aliased glyphs can be represented with very few grayscale values and therefore that the grayscale values may be represented in a quantized or compressed form by a small number of first index values without significant loss of information. For example, while each pixel of a source glyph representation may comprise a grayscale value represented by 8 bits (allowing 256 different grayscale values to be represented), only a few different grayscale values may be present in the source values. Accordingly, the 8-bit source values may be quantized to allow them to be represented by a first index occupying fewer bits without significant loss of information.

Similarly, index values used for encoding a visual effect, such as a blurring effect, may be represented with a small number of quantized values without degrading the visual impression compared to if they were represented by a larger number of bits. These values may therefore be encoded, via the second index, in a relatively small number of bits. Accordingly, the foreground and the background information may be encoded efficiently, with some of the available bits per pixel being used to store the first index representing the foreground information and the remaining bits used to store the second index representing the background information.

This allows the generated glyph representation to represent both the foreground and background information and to do so in a memory-efficient manner. For example, the source glyph representation may comprise 8 bits per pixel and the first indices and the second indices may be determined such that they together occupy fewer than or equal to 8 bits per pixel. Accordingly, the further glyph representation may occupy only minimal additional storage capacity compared to source glyph representation, with the additional storage capacity being due to the additional pixels in the further glyph representation representing the margin around the source pixels.

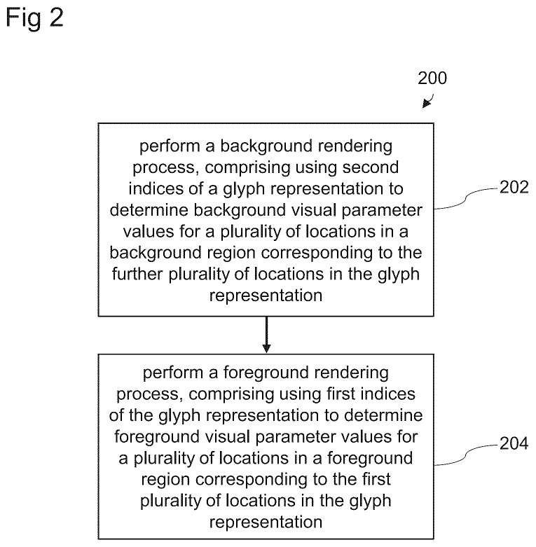

FIG. 2 is a flowchart illustrating an example computer-implemented method 200 of rendering a glyph using a glyph representation generated by the method 100 of FIG. 1.

The method 200 comprises, at block 202, performing a background rendering process. The background rendering process comprises using the second indices of the glyph representation generated by the method 100 to determine background visual parameter values for a plurality of locations in a background region. The plurality of locations in the background region correspond to the further plurality of locations in the glyph representation. The plurality of locations in the background region may, for example, be a plurality of pixels in a rendering of a background of the glyph representation. For example, the background region may comprise a set of pixels used to render a background shadow effect for a glyph on top of which the foreground of the glyph may be rendered in a second rendering pass, as described below. In one example, the background region may comprise a region of a medical image onto which text is to be rendered with a background effect.

Determining the background visual parameter values may comprise applying a pre-determined mapping between the second indices and background visual parameter values. For example, a look-up table may provide a mapping between values of the second indices and background visual parameter values. The second indices may be mapped to any visual parameter values suitable for providing a representation of the desired background effect. The background visual parameter values determined from the second indices may, for example, be grayscale values.

At block 202, the second indices may be mapped to grayscale values representing a shadow effect or any other desired effect. For example, where the second index represents a distance between a given pixel and one or more foreground pixels, a mapping between the second indices may be provided to generate a decaying shadow effect. For example, values of the second index which indicate close proximity to a foreground pixel may be mapped to low (i.e., dark) grayscale values while values of the second index which indicate greater distance from a foreground pixel are mapped to higher (i.e., lighter) grayscale values. The mapping may, for example, be linear to provide a linearly decaying shadow effect, or may be non-linear to provide a shadow effect which decays in any arbitrary manner.

In other examples, any desired background effect may be provided by appropriately mapping values of the second index to visual parameter values. For example, rather than a decaying shadow effect, a mapping may be provided which causes the generated background effect to be a sharp outline, an opaque rectangle or any other background effect.

At block 204, the method 200 comprises performing a foreground rendering process. The foreground rendering process comprises using the first indices of the glyph representation determined by the method 100 to determine foreground visual parameter values for a plurality of locations in a foreground region. The plurality of locations in the foreground region correspond to the first plurality of locations in the glyph representation determined by the method 100.

The foreground rendering process allows a foreground of the glyph represented by the glyph representation to be rendered on top of the background effect rendered at block 202. For example, at block 204, the black box of the glyph may be rendered on top of a shadow effect rendered by the background rendering pass of block 202.

In this manner, since the glyph representation generated by the method 100 encodes both foreground and background information of a glyph, it may be used in the method 200 to render both a background visual effect for the glyph and the foreground of the glyph. For example, a foreground of the glyph and a shadow effect may be rendered using the same glyph representation.

Since the glyph representation encodes both the foreground and the background visual effect information, visual effect information need not be calculated on-the-fly during rendering. Furthermore, there may be no need for an intermediate memory buffer into which glyphs are rendered in order to calculate visual effect information, as may be the case in some existing methods which render text in an intermediate buffer and compute a shadow effect on-the-fly in a separate rendering pass. This may reduce computational costs and may also reduce memory costs.

The foreground rendering process may have any of the features described above with reference to the background rendering process. In particular, the background rendering process may comprise determining the foreground visual parameter values for each of the plurality of locations in the foreground region according to a pre-determined mapping between the first indices and visual parameter values, such as grayscale values. This may involve the use of a look-up table mapping values of the first indices to visual parameter values. The first indices may be mapped to any suitable visual parameter values for rendering the foreground of the glyph representation. For example, where the determining of the first indices in the method 100 involves quantizing the pixel grayscale values of the black box of the source glyph representation, the foreground rendering process may involve mapping the first indices back to quantized grayscale values representing the black box. In other examples, the first indices may be mapped to different visual parameter values to provide a desired visual effect for the foreground of the rendering. The mapping between the first indices and visual parameter values may be different or the same as the mapping between the second indices and visual parameter values.

The use of pre-defined mappings between the first indices and second indices and respective visual parameter values allows for the benefits of pre-calculating the first indices and second indices while allowing for different rendering results to be achieved by exchanging the mappings as desired. For example, different background visual effects may be provided by exchanging the mapping between the second indices and visual parameter values, e.g., by exchanging a look-up table. Accordingly, for example, different shadow effects may be provided by using a different look-up table and without needing to execute a different shadow-effect algorithm. Similarly, different foreground rendering effects may be achieved by exchanging the look-up table used for mapping the first indices to visual parameter values.

Rendering using such pre-defined mappings may provide particular advantages in the context of medical rendering. For example, text can be rendered with visual effects which may be optimized to provide better legibility of text rendered on top of a medical images. The method allows this to be done in a computationally efficient manner which minimizes the effect on run-time performance. Moreover, a suitable visual effect for rendering text can be selected, or visual effects can be easily switched between, based on the current medical rendering task. For example, workflow mapping, user preferences, image metadata (e.g. DICOM data), hanging protocol or device DPI properties may be used to select a pre-defined mapping which provides an appropriate visual effect for the medical rendering task at hand.

Furthermore, since the visual effect information is not calculated on-the-fly during a rendering process, but instead is pre-calculated and encoded in the second indices, the overall rendering result may be produced in a more device-independent manner. For example, if a shadow effect algorithm is computed on-the-fly during rendering, the resulting visual impression may differ between different devices due to, for example, how the different devices handle arithmetic rounding operations. This may be particularly valuable in applications where deterministic renderings are desirable for quality reasons, such as in medical rendering applications. For example, since the results of a medical rendering operation may commonly be reviewed by several experts, any differences in visual impression should be minimized or avoided to help minimize confusion.

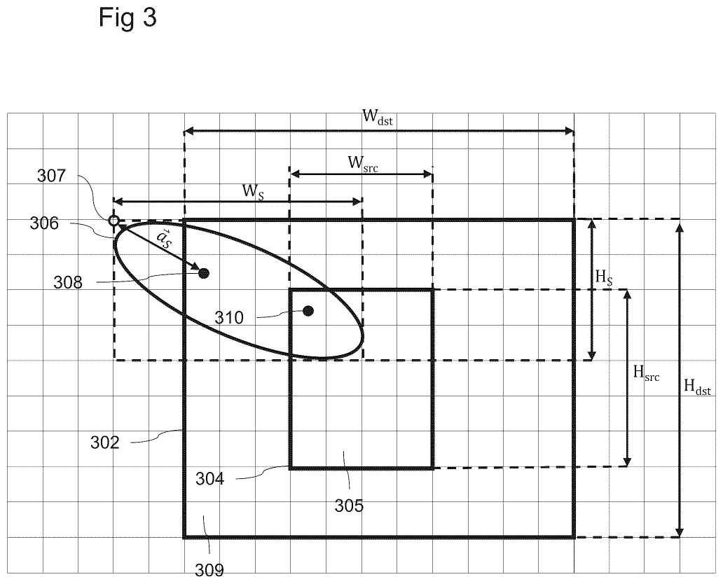

Turning to FIG. 3, there is shown a schematic drawing illustrating aspects of an example method for generating a glyph representation according to the method of FIG. 1.

FIG. 3 shows schematically a glyph representation 302 which in this example is referred to as a destination glyph representation. The destination glyph representation 302 has a height Hdst and a width Wdst, each measured as a number of pixels. In this example, Hdst=9 and Wdst=11.

The destination glyph representation 302 includes a source region 304 having a height of Hsrc of 5 pixels and a width of Wsrc of 4 pixels. The pixels 305 within the source region 304 may be referred to as foreground pixels 305 since they correspond with the source pixels of a source glyph representation (not shown) based upon which the destination glyph representation 302 is generated. Surrounding the source region 304 are additional pixels 309 within the destination glyph representation 302. These additional pixels 309 provide a margin of pixels around the source region 304 allowing a background effect, such as a blurred shadow, to be represented by the destination glyph representation 302.

The positioning of the additional pixels 309 around the source region 304 is determined by a shadow neighborhood 306. The shadow neighborhood 306 may be considered to determine the size and shape of a shadow produced by a given foreground pixel. The shadow neighborhood 306 is defined with a given shape and size which in examples may be selected in an arbitrary manner depending on the desired shadow effect to be achieved. The shadow neighborhood 306 has a width WS and a height HS, each measured as a number of pixels. In this example, WS=7 while HS=4.

The width Wdst of the glyph representation 302 is related to the respective widths Wsrc and WS of the source region 304 and the shadow neighborhood 306 by Wdst=Wsrc+WS. Similarly, the height Hdst of the glyph representation 302 is related to the respective heights Hsrc and HS of the source region 304 and the shadow neighborhood 306 by Hdst=Hsrc+HS. An anchor point 307 and an anchor point offset \vec{a}_{S} is defined for the shadow neighborhood 306 which determines the position of the shadow neighborhood 306 with respect to a given destination pixel 308 being encoded.

To generate the destination glyph representation 302, the following steps are performed:

A destination glyph representation memory buffer is initialized with a number Bdst of bits per pixel. Bdst may, for example, be 4, 8 or 16. In some examples, Bdst may be equal to a number Bsrc of bits per pixel of the source glyph representation. For example, both the source glyph representation and the destination glyph representation 302 may comprise 8 bits per pixel.

For each given pixel 308 having co-ordinates Post within the glyph representation 302:

The shadow neighborhood is positioned with its anchor point 307 at P_{dst}−\vec{a}_{S}.

A relationship between the given pixel 308 and one or more pixels of the foreground pixels 305 which lie within the shadow neighborhood 306 is determined. FIG. 3 shows a foreground pixel 310 which is within the shadow neighborhood 306 and, in this example, a relationship between the given pixel 308 being encoded and the foreground pixel 310 is determined. The relationship may be geometric relationship, for example a distance metric, such as a Euclidean or Manhattan distance between the given pixel 308 and the foreground pixel 310. This determined relationship is used to set an index Is.

Where the determined relationship is a distance between the pixel 308 and the foreground pixel 310, the value of Is may be set to be proportional to the determined distance.

In some examples, the index Is for a given pixel 308 may be set independently of the source value of the foreground pixels within the shadow neighborhood 306. For example, the index Is may be set solely based on a geometric relationship between the given pixel 308 and the foreground pixels within the shadow neighborhood 306.

In other examples, the value of Is may also be set based on a visual parameter value, e.g. grayscale value, of the source pixel corresponding with the foreground pixel 310. For example, a factor proportional to the distance between 308 and 310 may be multiplied by the grayscale value of the source pixel corresponding to the foreground pixel 310 to provide a resultant grayscale value. This resultant grayscale value may be used to set the index Is from a set of index values.

In one such example, a number Nf of quantization bins is defined within a range [0,2^{B}^{_{src}}]. The quantization bins may be distributed in any way, for example, regularly or irregularly, within this range. In one simplified example, for 8-bit grayscale values ranging from 0 to 255, four quantization bins may be defined, e.g. bin 0:0-45, bin 1:46-85, bin 2:86-200, bin 3:201-255. A determination is made of the quantization bin into which the resultant grayscale value falls, and the index Is is set as the label of this quantization bin, e.g. 0, 1, 2 or 3 in the above example where four quantization bins are defined. This example is represented in Table 1. In this example, a resultant grayscale value of 89 would fall into the quantization bin with label 2 resulting in a value of Is=2.

TABLE 1

| Quantization | Grayscale |
|---|---|
| bin | values |
| 0 | 0-45 |
| 1 | 46-85 |
| 2 | 86-200 |
| 3 | 201-255 |

In examples where there is more than one foreground pixel 305 within the shadow neighborhood 306, the index Is may be determined based on a relationship between the given pixel 308 and each of the foreground pixels 305 which are within the shadow neighborhood 306. For example, grayscale values representing respective shadow contributions at the pixel 308 from each of the foreground pixels 305 which are within the shadow neighborhood 306 may be calculated in the manner described above and combined, e.g. by alpha blending, to determine an overall resultant grayscale value for the pixel 308. This overall resultant grayscale value may then be used to select the index Is from the set of index values, for example, by a quantization process as described above.

In this manner, the index Is can be used to encode a shadow effect value for the given pixel 308. This shadow effect value can be used, as will be described in more detail below, when rendering a background of the glyph represented by the glyph representation 302.

To set an index, If, a determination is made of whether the given pixel 308 is within the source region 304. If the given pixel 308 is within the source region 304, then the source value, e.g. grayscale value, of the source pixel corresponding with the given pixel 308 is used to set the index If.

In one example, a number of quantization bins is defined and a determination is made of the quantization bin into which the source value falls. The index If is set as the label of this quantization bin. The definition of the quantization bins may, for example, be determined based on the grayscale values which are present in the source values. For example, where only a few grayscale values are present in the source values, which may often be the case for the black box text of a glyph, then a small number of suitably selected quantization bins may be used to represent the source values by the index If without significant loss of visual information. In this manner, the source value of the source pixel corresponding to the given pixel 308 may be represented in a quantized or compressed manner by the value of the index If.

The definition of the quantization bins, e.g. the number of bins of the range of grayscale values in each bin, may be the same or different to for the quantization bins used to set the index Is in the above example where such quantization bins are used to set the index Is. In certain examples, the shadow effect values represented by the index Is, on the other hand, may benefit from allowing a larger number of grayscale values to be represented. For example, whereas the black box text of a glyph may be represented by only 3 or 4 grayscale values, it may be advantageous to represent a shadow effect using a larger number of grayscale values, in order to provide a visually smooth effect. In such examples, a larger number of bits may be used to store the index Is compared to the number of bits used to store the index If.

If the pixel 308 is determined not to be within the source region 304 (such as for the example shown in FIG. 3) then the index If may be set to indicate this. For example, the index If may be set with a void value or a value of 0.

The index Is is stored in Bs bits associated with the given pixel 308 while the index If is stored in Bf bits associated with the given pixel 308 which are not used for storing the index Is. The total number of bits per pixel for the destination glyph representation Bdst is such that (B_{f}+Bₛ)≤B_{dst}. In an example, the indices Is and If for a given pixel 308 are stored as a single pixel value V.

This procedure may be performed to encode each of one or more glyphs in respective destination glyph representations. The destination glyph representations may be stored in a single glyph atlas by positioning the individual destination glyph representations with respect to one another in an appropriate manner using any suitable bin packing algorithm.

In an example, in order to render a glyph using the destination glyph representation 302 of FIG. 3, the following steps are performed.

In a first rendering pass, a background of the glyph, in this example, providing a shadow effect, is rendered. In this first rendering pass, for each pixel in the rendering of the glyph, the pixel value V of the corresponding pixel 308 in the destination glyph representation 302 is read. From the value V, the index Is is decoded. This may be done by bit shifting and bit masking such that only the Bs bits representing the index Is remain.

The index Is is then used to obtain a shadow effect grayscale value

$${V}_{s}^{′}$$

for the pixel in the rendering using a look-up table Ts, i.e.

$${V}_{s}^{′} = {T}_{s} [ {I}_{s} ] .$$

The look-up table Ts is of size Nₛ ∈[1, 2^{B}^{ₛ}]. For example, if the number of bits Bs for storing the index Is is 2, then the index Is may take up to 4 different values. Each of these different values of Is may be mapped, via a row in the look-up table Ts, to a shadow effect grayscale value. The grayscale values to which the values of the index Is are mapped may be any suitable values for providing the desired shadow effect. For example, to provide a linearly decaying shadow effect, where increasing values of the index Is represent increasing distance from a foreground pixel, a linear mapping may be defined such that increasing values of the index Is are mapped to increasing grayscale values (i.e. lighter shades of grayscale values).

As described above, while the shadow neighborhood 306 may be considered to determine the size and shape of a shadow produced by a given foreground pixel, depending on the mapping between values of the second index Is and grayscale values, the size and shape of the shadow may not entirely correspond with the shadow neighborhood 306. For example, the rendered shadow may appear smaller than the shadow neighborhood 306 if, due to the mapping between values of the second index Is and grayscale values, the outermost pixels in the rendered background region are completely transparent.

In another example, in the example where the second indices are set by determining a quantization bin into which a computed shadow effect grayscale value falls, a look-up table may map the second index to a grayscale value related to the quantization bin used to set the second index value. For example, in the example of Table 1, each value of the second index may be mapped by the look-up table to the lowest grayscale value of the corresponding quantization bin, as is shown in Table 2.

TABLE 2

| | Grayscale |
|---|---|
| Iₛ | value |
| 0 | 0 |
| 1 | 46 |
| 2 | 86 |
| 3 | 201 |

In other examples, values of the index Is may be mapped to any suitable grayscale or other visual parameter values. Further, by exchanging the mapping, the visual effect provided may be changed. Any further coloring or additional effects may be applied using the grayscale value

$${V}_{s}^{′}$$

as an input to obtain a final pixel value for the background pixel in the rendering of the glyph.

In a second rendering pass the foreground of the glyph is rendered. The second rendering pass is similar to the first rendering pass in that for each pixel in the rendering of the glyph, the pixel value V of the corresponding pixel 308 in the glyph representation 302 is read. From the value V, the index If is decoded, which may, similarly as described above for the index Is, involve performing a bit shifting and bit masking operation on the value V until only the bits Bf representing the index If remain.

The index If is then mapped to obtain a foreground grayscale value

$${V}_{f}^{′}$$

for the pixel in the rendering using a look-up table Tf, i.e.

$${V}_{f}^{′} = {T}_{f} [ {I}_{f} ] .$$

The look-up table Tf is of size N_{f}∈[1,2^{B}^{_{f}}], similarly to as described for the look-up table Ts. The relative sizes of the look-up tables Ts and Tf depend on the relative sizes of the number of bits Bs used to encode the index Is compared with the number of bits Bf used to encode the index If. The mapping provided by the look-up table Tf may be the same or different to the mapping provided by the look-up table Ts. Different foreground rendering effects may be provided by exchanging the look-up table Tf used to map the indices If to grayscale values. As with the shadow effect grayscale value

$${V}_{s}^{′} ,$$

any further coloring or additional effects may be applied using the grayscale value

$${V}_{f}^{′}$$

as an input to obtain a final pixel value for the pixel in the rendering. The grayscale values

$${V}_{s}^{′} and {V}_{f}^{′}$$

may be combined in any suitable manner to provide a final pixel value for the rendering. For example, the values may be blended by alpha blending or any suitable process.

The look-up tables Ts and Tf may be defined to provide any suitable mapping of values of the indices Is and If to visual parameter values. In some examples, multiple look-up tables may be defined for either or both of the indices Is and If allowing the look-up table being used to be exchanged, for example, dependent on the rendering task being performed. For example, as described above, different background visual effects may be provided by exchanging the look-up table Ts used to map the indices Is to grayscale values. As an example, as described above, in a medical rendering application, the look-up table Ts may be selected based on properties of the medical rendering task being performed, such as workflow mapping, user preferences, image metadata (e.g. DICOM data), hanging protocol or device DPI properties.

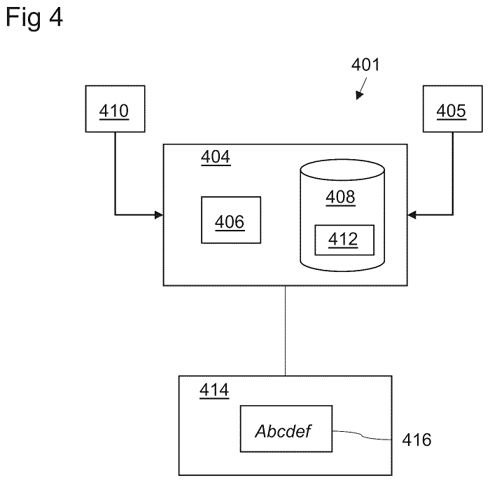

FIG. 4 is a schematic drawing illustrating an example system 401 in which an example apparatus 404 may use methods described herein. The system 401 comprises the apparatus 404 and a visualization unit 414. In examples, the system 401 may comprise fewer components than or additional components to those illustrated in FIG. 4. For example, the system 401 may comprise a computer network such as the internet.

The apparatus 404 comprises one or more processors 406 and a memory, in the form of one or more storages 408. The apparatus 404 may, for example, comprise a GPU. In this example, the apparatus 404 is arranged to perform a method according to examples described above of generating a glyph representation and a method according to examples described above for rendering a glyph using such a glyph representation.

The storage 408 may comprise a machine-readable medium comprising a set of machine-readable instructions which when executed by the processor 406 cause the apparatus 404 to perform an example method described herein. The program may be stored on a computer readable medium which may be read by the apparatus 404 to thereby execute the program.

The apparatus 404 may be arranged to receive directly or indirectly or otherwise acquire a source glyph representation 410 and generate based on the source glyph representation 410 a destination glyph representation 412 to be stored on the storage 408. The source glyph representation 410 may form a part of a source glyph atlas (not shown) comprising a plurality of source glyph representations. Similarly, the destination glyph representation 412 may form a part of a destination glyph atlas (not shown) comprising a plurality of destination glyph representations.

The apparatus 404 may also be configured to receive directly or indirectly or otherwise acquire one or more respective look-up tables 405 for mapping values of the first and second indices in the destination glyph representation 412 to visual parameter values, e.g. grayscale values. The look-up tables 405 may comprise one or more look-up tables for the first indices and one or more look-up tables for the second indices. For example, multiple look-up tables for mapping the second indices to grayscale values may be provided to allow different visual parameter effects to be generated. The apparatus 404 may be configured to select one of these look-up tables to use during the rendering based on the context of the rendering. Alternatively, or additionally, the apparatus 404 may be configured to generate one or more look-up tables, for example, based on source values of the source glyph representation 410 and/or one or more visual-effect calculations.

The apparatus 404 may also be arranged to generate, using the destination glyph representation 412, a rendering of a glyph including both background and foreground visual information. For example, the apparatus 404 may be configured to generate using the destination glyph representation 412 and one or more further destination glyph representations in a glyph atlas a rendering of text including a background shadow effect. The apparatus 404 may be arranged to transmit information, for example, visual parameter information, e.g. grayscale of color values, representing the rendering to a visualization unit 414. The transmission may be direct or indirect, for example via a wired connection, a wireless connection, or via the internet.

The visualization unit 414 may comprise visualization software for displaying the rendering produced by the apparatus 404. For example, text 416 rendered by the apparatus 404 may be displayed by the visualization unit 414. The visualization unit 414 may comprise a display screen, and one or more graphics hardware or software components. In some examples, the visualization unit 414 may be or comprise a mobile device. In some examples the visualization unit 414 may comprise a virtual reality or augmented reality device. The visualization unit 414 may in some examples display a stereo image.

In some examples, the system 401 is a system for rendering medical images with the rendered text 416 being displayed over the medical image. In such examples, the apparatus 404 may also be arranged to generate a rendering of a medical dataset, for example, a volume rendering of a volumetric dataset and transmit information representing the medical image to the visualization unit 414. In some such examples, the system 401 may also comprise a scanner (not shown) for generating a medical dataset, which may, for example, be a volumetric dataset representing a portion of a patient. The scanner may be a computed tomography (CT) scanner, a magnetic resonance imaging (MRI) scanner, a positron emission tomography (PET) scanner, an ultrasound scanner or the like. In such examples, the scanner may be connected to the apparatus 404, for example via wired or wireless connection and may be arranged to provide the medical dataset to the apparatus 404 for rendering.

While in certain examples described above, the destination glyph representation comprises a single first index representing foreground information and a single second index representing background information, in other examples, there may be more than one first index and/or more than one second index per pixel. For example, where the source glyph representation is a color representation, each pixel in the source glyph representation may comprise more than one color value, e.g. respective R, G and B values may be stored per pixel. In such an example, three first indices may be stored in association with each pixel in the further glyph representation, the first indices at the location representing, respectively R, G and B color values. Each of these values may be generated and stored in association with the pixel in the manner described above. When rendering using the glyph representation, each of the first indices may be used to determine a respective visual parameter value (e.g. R, G or B color value) for the given pixel according to a pre-determined mapping, such as by using a look-up table. Similarly, there may be more than one second index per pixel in the destination glyph representation, where each of these values may also be generated and stored in association with the pixel in the manner described above. This may, for example, provide for a background effect to also be encoded in a manner which represents color, rather than grayscale, visual information.

The above embodiments are to be understood as illustrative examples of the present invention. Other embodiments are envisaged. It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the present invention, which is defined in the accompanying claims.

Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A computer-implemented method of generating, based on a source glyph representation, a further glyph representation for use in a rendering process, the computer-implemented method comprising:
determining a first plurality of locations in the further glyph representation corresponding with a respective plurality of source locations in the source glyph representation;
setting respective first indices for the first plurality of locations based on values associated with the respective plurality of source locations;
setting, for each location of a further plurality of locations in the further glyph representation, respective second indices based on a relationship between the location and one or more source locations in the source glyph representation; and
storing the respective first indices and the respective second indices in association with respective locations.

2. The computer-implemented method of claim 1, wherein the setting of the respective first indices comprises:
applying a quantization process to the values associated with the respective plurality of source locations.

3. The computer-implemented method of claim 1, wherein the relationship comprises a geometric relationship between the location and one or more of the one or more source locations.

4. The computer-implemented method of claim 3, wherein the geometric relationship comprises a distance metric.

5. The computer-implemented method of claim 1, wherein the setting of the respective second indices comprises:
performing a visual-effect calculation based on the relationship between the location and the one or more source locations in the source glyph representation.

6. The computer-implemented method of claim 5, wherein the visual-effect calculation comprises at least one of a shadow-effect calculation or a blurring-effect calculation.

7. The computer-implemented method of claim 1, wherein
the respective first indices and the respective second indices are stored in a number of bits per location in the further glyph representation, and
the number of bits per location in the further glyph representation is less than or equal to a number of bits per location in which the values associated with the respective plurality of source locations are stored in the source glyph representation.

8. The computer-implemented method of claim 1, wherein the values associated with the respective plurality of source locations are visual parameter values.

9. A computer-implemented method of rendering a glyph generated according to the computer-implemented method of claim 1, the computer-implemented method comprising:
performing a background rendering process, the background rendering process including using the respective second indices to determine background visual parameter values for a plurality of locations in a background region corresponding to the further plurality of locations in the further glyph representation; and
performing a foreground rendering process, the foreground rendering process including using the respective first indices to determine foreground visual parameter values for a plurality of locations in a foreground region corresponding to the first plurality of locations in the further glyph representation.

10. The computer-implemented method of claim 9, wherein at least one of
determining of the background visual parameter values for the plurality of locations in the background region is based on a mapping between the respective second indices and the background visual parameter values, or
determining of the foreground visual parameter values for the plurality of locations in the foreground region is based on a mapping between the respective first indices and the foreground visual parameter values.

11. The computer-implemented method of claim 10, wherein the computer-implemented method of rendering the glyph is applied in a medical rendering process, and wherein the computer-implemented method further comprises:
selecting at least one of the mapping between the respective second indices and the background visual parameter values or the mapping between the respective first indices and the foreground visual parameter values based on a property of the medical rendering process.

12. A non-transitory machine-readable medium comprising one or more glyph representations generated by the computer-implemented method according to claim 1.

13. A non-transitory machine-readable medium comprising a set of machine-readable instructions that, when executed by a processor, cause the processor to perform the computer-implemented method according to claim 1.

14. An apparatus comprising:
a processor; and
a memory including one or more glyph representations generated by the computer-implemented method according to claim 1.

15. An apparatus comprising:
a processor; and
a memory including machine-readable instructions that, when executed by the processor, cause the apparatus to perform the computer-implemented method according to claim 1.

16. The computer-implemented method of claim 2, wherein the relationship comprises a geometric relationship between the location and one or more of the one or more source locations.

17. The computer-implemented method of claim 8, wherein the visual parameter values are grayscale or color values.

18. The computer-implemented method of claim 2, wherein the setting of the respective second indices comprises:
performing a visual-effect calculation based on the relationship between the location and the one or more source locations in the source glyph representation.

19. The computer-implemented method of claim 3, wherein the setting of the respective second indices comprises:
performing a visual-effect calculation based on the relationship between the location and the one or more source locations in the source glyph representation.

20. The computer-implemented method of claim 2, wherein
the respective first indices and the respective second indices are stored in a number of bits per location in the further glyph representation, and
the number of bits per location in the further glyph representation is less than or equal to a number of bits per location in which the values associated with the respective plurality of source locations are stored in the source glyph representation.
